(19) United States
(12) Patent Application Publication
(10) Pub. No.: US 2013/0304129 A1
(43) Pub. Date: Nov. 14, 2013

(54) SURGICAL HOOK INCLUDING FLOW PATH

(71) Applicant: ALPHATEC SPINE, INC., Carlsbad, CA (US)

(72) Inventors: Harold HAWKINS, Carlsbad, CA (US); Darren REIMERS, Carlsbad, CA (US); S. Samuel BEDERMAN, Orange, CA (US)

(73) Assignee: Alphatec Spine, Inc., Carlsbad, CA (US)

(21) Appl. No.: 13/874,978

(22) Filed: May 1, 2013

Related U.S. Application Data
(60) Provisional application No. 61/643,196, filed on May 4, 2012.

(51) Int. Cl. A61B 17/70

(52) U.S. Cl. CPC A61B 17/7056
USPC 606/276

(57) ABSTRACT

A hook for attaching a surgical rod to a vertebra includes a rod-receiving portion, a base portion, and a tip portion. The rod-receiving portion includes a pair of side walls that form a channel for receiving the surgical rod. The base portion extends distally from the rod-receiving portion and includes a first flow path extending therethrough. The tip portion extends away from the rod-receiving portion to form a hook portion for attaching to the vertebrae. In other features, the hook includes a second flow path extending through the tip portion.

### CROSS-REFERENCE TO RELATED APPLICATIONS

The present application claims priority to U.S. Provisional App. Ser. No. 61/643,196 filed on May 4, 2012, entitled “Surgical Hook Including Flow Path” which is incorporated herein by reference in its entirety.

### FIELD

The present disclosure generally relates to the field of spinal orthopedics, and more particularly to systems and methods for spinal fixation.

### BACKGROUND

The spine is a flexible column formed of a plurality of bones called vertebrae. The vertebrae are hollow and piled one upon the other, forming a strong hollow column for support of the cranium and trunk. The hollow core of the spine houses and protects the nerves of the spinal cord. The different vertebrae are connected to one another by means of articular processes and intervertebral, fibrocartilaginous bodies. Various spinal disorders may cause the spine to become misaligned, curved, and/or twisted or result in fractured and/or compressed vertebrae. It is often necessary to surgically correct these spinal disorders.

The spine includes seven cervical (neck) vertebrae, twelve thoracic (chest) vertebrae, five lumbar (lower back) vertebrae, and the fused vertebrae in the sacrum and coccyx that help to form the hip region. While the shapes of individual vertebrae differ among these regions, each is essentially a short hollow shaft containing the bundle of nerves known as the spinal cord. Individual nerves, such as those carrying messages to the arms or legs, enter and exit the spinal cord through gaps between vertebrae.

The spinal disks act as shock absorbers, cushioning the spine, and preventing individual bones from contacting each other. Disks also help to hold the vertebrae together. The weight of the upper body is transferred through the spine to the hips and the legs. The spine is held upright through the work of the back muscles, which are attached to the vertebrae. While the normal spine has no side-to-side curve, it does have a series of front-to-back curves, giving it a gentle “S” shape. If the proper shaping and/or curvature are not present due to scoliosis, neuromuscular disease, cerebral palsy, or other disorder, it may be necessary to straighten or adjust the spine into a proper curvature.

Generally the correct curvature is obtained by manipulating the vertebrae into their proper position and securing that position with a rigid system of screws and rods. The screws may be inserted into the pedicles of the vertebrae to act as bone anchors, and the rods may be inserted into receiving heads of the screws. Two rods may run substantially parallel to the spine and secure the spine in the desired shape and curvature. Thus the rods, which are shaped to mimic the correct spinal curvature, force the spine into proper alignment. Bone grafts are then placed between the vertebrae and aid in fusion of the individual vertebrae together to form a correctly aligned spine.

In some instances, screws may not be ideal or appropriate for securing the rods within the system. Often, surgical hooks including receiving heads similar to the screws may be used in place of the screws to attach the rods to portions of the vertebrae. In some cases, portions of the vertebrae may be removed to position the hooks and to enhance bone growth around the hooks. These hooks may be used within the cervical, thoracic, and lumbar regions of the spine. The hooks may attach to lamina, transverse processes, pedicles, and other portions of the vertebrae. The hooks may include various orientations relative to the receiving heads to accommodate various orientations and curvatures of the spine.

Generally bone fusion materials may be used with the screws, rods, and spacers to facilitate fusion of the vertebrae. However, surgical hooks may not be immersed in growing bone or bone fusion material. This may lead to a less rigid construct since the boney material may not sufficiently grow around the hooks to retain them in place after a fusion procedure. Therefore, one aspect of the inventions of the present disclosure attempts to improve fusion of the spine using surgical hooks.

### SUMMARY

A hook for attaching a surgical rod to a vertebra includes a rod-receiving portion, a base portion, and a tip portion. The rod-receiving portion includes a pair of side walls that form a channel for receiving the surgical rod. The base portion extends distally from the rod-receiving portion and includes a first flow path extending therethrough. The tip portion extends away from the rod-receiving portion to form a hook portion for attaching to the vertebrae. In other features, the hook includes a second flow path extending through the tip portion.

In still other features the first flow path includes a window. The first flow path includes a plurality of thru-holes. The first flow path includes a mesh screen.

A hook for attaching a surgical rod to a vertebra includes a rod-receiving portion including a pair of side walls that form a channel for receiving the surgical rod and a bone receiving portion extending distally from the rod-receiving portion and forming a hook, the hook including at least one flow path for enhanced flow of bone material through the hook during bone fusion.

### BRIEF DESCRIPTION OF THE DRAWINGS

FIG. 1 is a perspective view of a spinal fixation system including various rods, cross-links, and hooks according to the principles of the present disclosure.

FIG. 2 is a perspective view of a rod and pair of hooks according to the principles of the present disclosure.

FIG. 3 is a lateral side view of an exemplary hook according to the principles of the present disclosure.

FIGS. 4A-4E are rear views of exemplary hooks according to the principles of the present disclosure.

FIG. 5 is a lateral side view of another exemplary hook according to the principles of the present disclosure.

FIG. 6 is a rear side view of the hook of FIG. 5 according to the principles of the present disclosure.

FIG. 7 is a bottom side view of the hook of FIG. 5 according to the principles of the present disclosure.

### DETAILED DESCRIPTION

The devices and methods of the present disclosure include improvements to surgical hooks to further enhance arthrodesis or bone growth for fusion surgeries. Exemplary surgical hooks include rod receiving heads and hooks. The hooks of the present disclosure may include various flow paths such as openings, windows, slots, holes, apertures, and the like to improve flow of bone fusion materials such as bone cement, autograft, allograft, and the like. The flow paths may be incorporated in the hook portion of the surgical hooks.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein. The words proximal and distal are applied herein to denote specific ends of components of the instrument described herein. A proximal end refers to the end of an instrument nearer to an operator of the instrument when the instrument is being used. A distal end refers to the end of a component further from the operator and extending towards the surgical area of a patient and/or the implant.

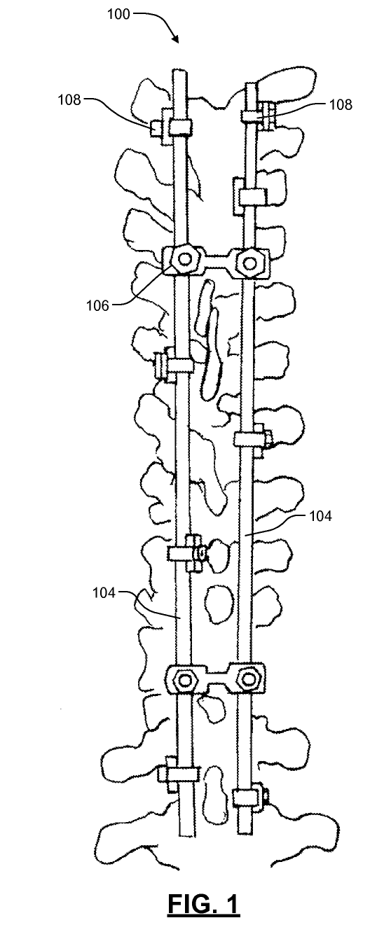

Referring now to FIG. 1, a spinal fixation system 100 may include any combination of screws, rods 104, cross-links 106, and hooks 108 for attachment to vertebrae of the spinal column. Although the exemplary system 100 of FIG. 1 may be most suitable for use within the cervical-thoracic region of the spine, the devices of the present disclosure may be applicable to any region of the spine where hooks 108 may be used.

In FIG. 2, the hook 108 includes a rod-receiving portion 110 and a bone engagement or hook portion 112. The rod-receiving portion 110 may include a pair of side portions 114 that form a generally U-shaped channel for receiving the rod 104. The side portions 114 may include internal threads that mate with threads 116 of a setscrew 118. The setscrew 118 may secure the rod 104 within the rod-receiving portion 110. The hook portion 112 may be attached to bone portions of a vertebra by wrapping around the bone. For example, the hook portion 112 may attach to one of the articular processes of a vertebra.

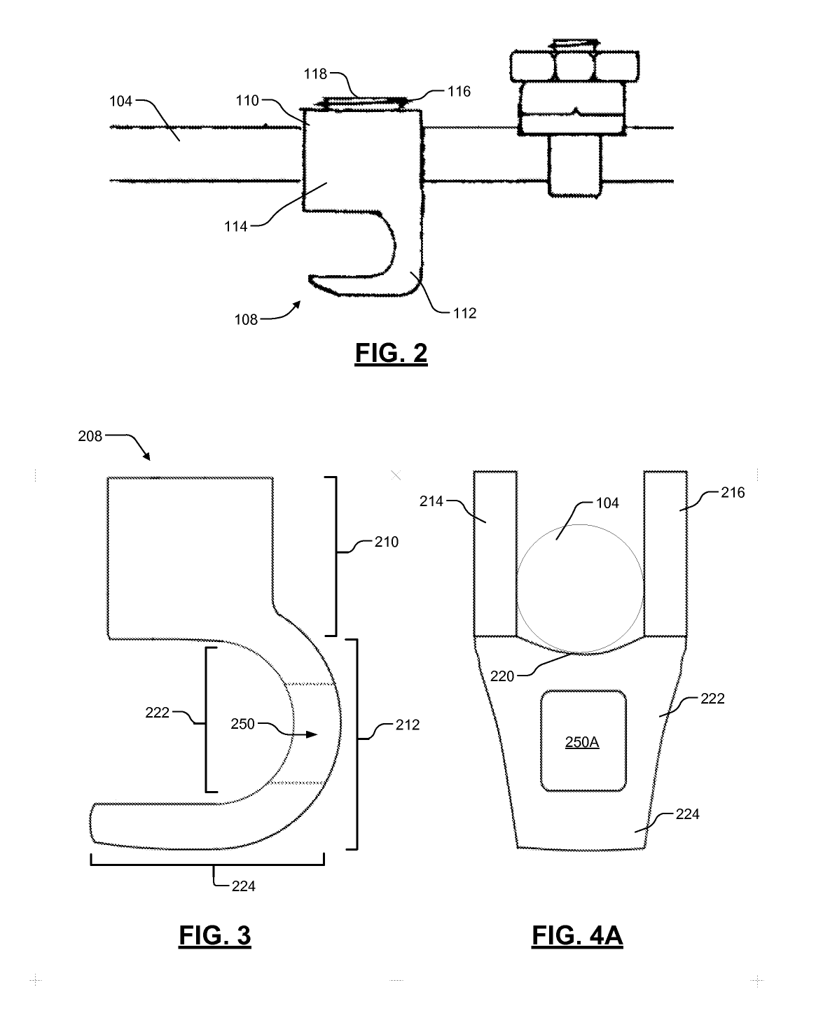

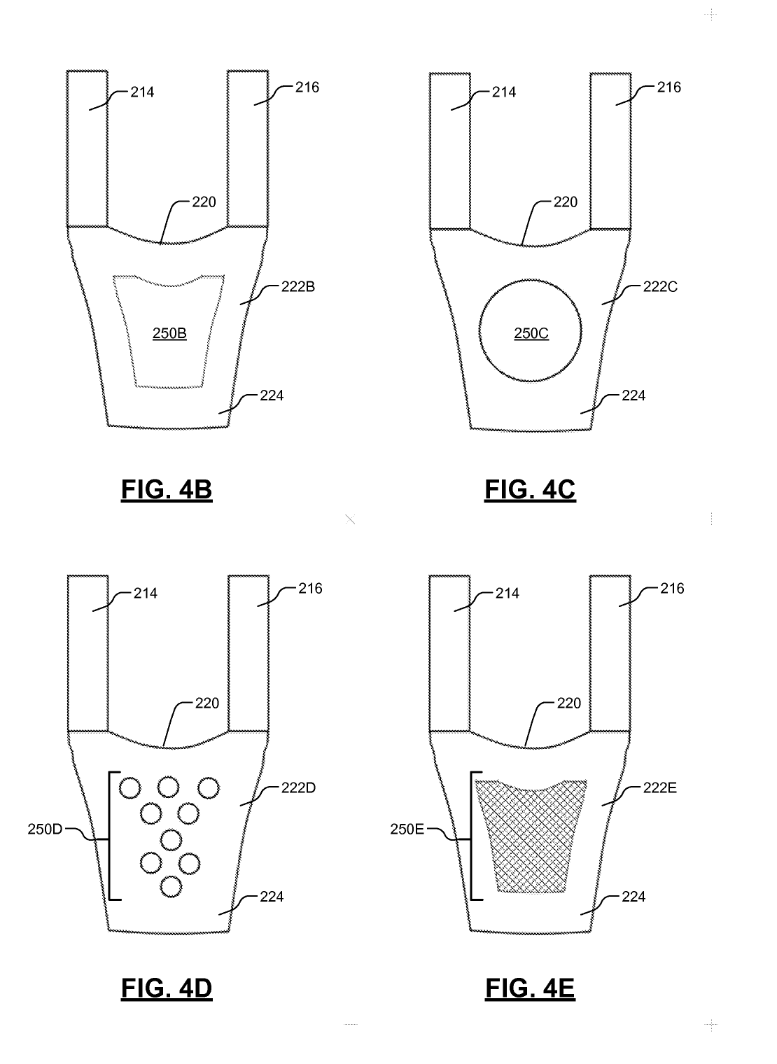

In order enhance flow of bone material for fusion, an exemplary hook 208 according to the principles of the present disclosure, includes one or more flow paths 250 as illustrated in FIGS. 3-4E. In each of the examples, the hooks 208 include rod-receiving portions 210 and hook portion 212. The rod-receiving portions 210 may be comprised of side portions 214 and 216. The side portions 214 and 216 form U-shaped channels 220 for receiving setscrews 118 as shown in FIG. 2. The hook portions 212 may include the flow paths 250 that extend through the hook portions 212 as illustrated in FIGS. 3.

Referring now to FIG. 3, the hook portion 212 may include a base portion 222 and a tip portion 224. The base portion 222 extends distally from the rod-receiving portion 210. The base portion 222 may include a uniform thickness as illustrated in FIG. 3. The base portion 222 may include a ramped thickness. The base portion 222 may include a radius of curvature. The tip portion 224 extends away from the base portion 222 to define the hook portion 212.

In FIG. 3, the flow path 250 takes the form of a window 250A extending through the base portion 222 of the hook 208 as illustrated by the dashed lines. The window 250A may include any shape. For example, in FIG. 4A, the window 250A includes a generally rectangular shape. In FIG. 4B, the window 250B may include an irregular shape. The window 250B may conform to the outer dimensions of the hook portion 212. The base portion 222B may be formed from rod-stock bent into a continuous loop to form the window 250B and affixed to the rod-receiving portion 210. In FIG. 4C, the window 250C forms a circular shape. The window 250C may be punched or cut from the base portion 222C. In FIG. 4D, a plurality windows or apertures 250D may be drilled or punched from the base portion 222D. In FIG. 4E, the window 250E may include flow guides, such as a mesh screen 260 for directing the flow of the bone material.

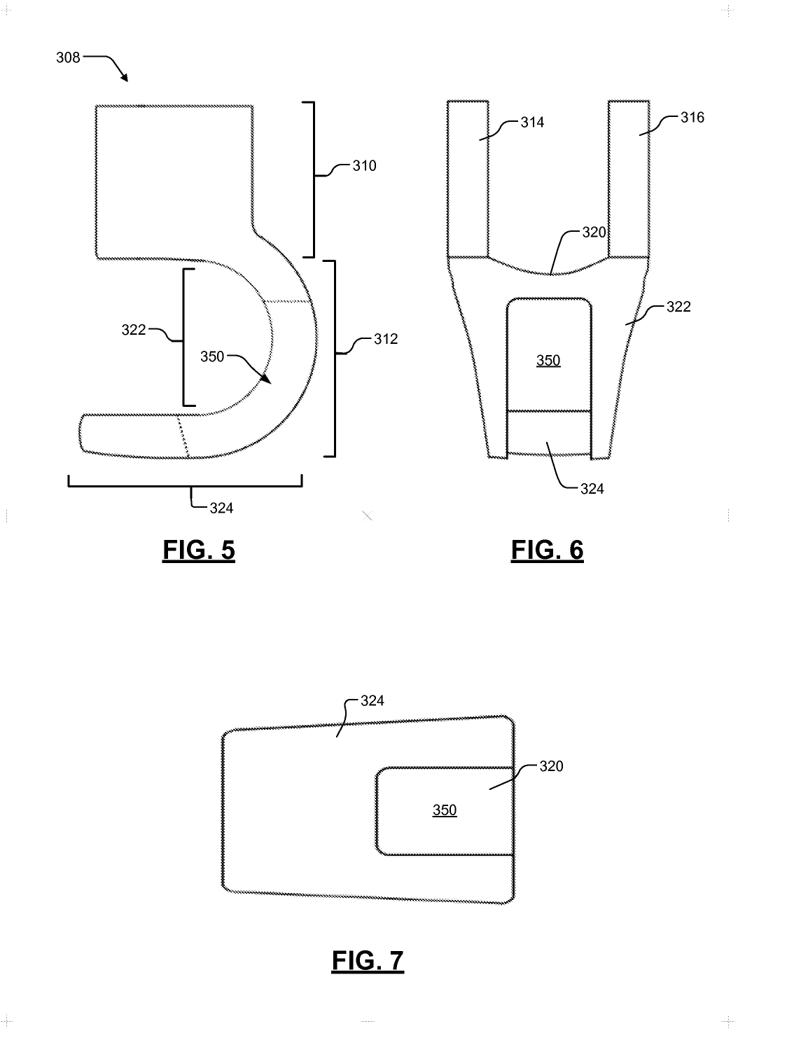

In order enhance flow of bone material for fusion, another exemplary hook 308 according to the principles of the present disclosure, includes one or more enlarged flow paths 350 as illustrated in FIGS. 5-7. In each of the examples, the hooks 308 include rod-receiving portions 310 and hook portion 312. The rod-receiving portions 310 may be comprised of side portions 314 and 316. The side portions 314 and 316 form U-shaped channels 320 for receiving setscrews 118 as shown in FIG. 2. The hook portions 312 may include a flow path 350 that extends through the hook portions 312, including a base portion 322 and a tip portion 324 as illustrated in FIG. 5.

Referring now to FIG. 5, the hook portion 312 may include the base portion 322 and the tip portion 324. The base portion 322 extends distally from the rod-receiving portion 310. The base portion 322 may include a uniform thickness as illustrated in FIG. 5. The base portion 322 may include a ramped thickness. The base portion 322 may include a radius of curvature. The tip portion 324 extends away from the base portion 322 to define the hook portion 312.

In FIG. 5, the flow path 350 takes the form of a window 350 extending through the base portion 322 and the tip portion 324 as illustrated by the dashed lines. The window 350 may include any shape. For example, in FIG. 6, the window 350 includes a generally rectangular shape. However, the window 350 may include any shape as described with reference to FIGS. 4A-4E. The window 350 wraps around the base portion 322 to extend through a portion of the tip portion 324 as illustrated in FIGS. 6 and 7.

Example embodiments of the methods and systems of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments, but should be defined only in accordance with the following claims and their equivalents.

The invention claimed is:

1. A hook for attaching a surgical rod to a vertebra, comprising:
a rod-receiving portion including a pair of side walls that form a channel for receiving the surgical rod;
a base portion extending distally from the rod-receiving portion and including a first flow path extending therethrough; and
a tip portion extending away from the rod-receiving portion to form a hook portion for attaching to the vertebrae.

2. The hook of claim 1, further comprising a second flow path extending through the tip portion.

3. The hook of claim 1, wherein the first flow path includes a window.

4. The hook of claim 1, wherein the first flow path includes a plurality of thru-holes.

5. The hook of claim 1, wherein the first flow path includes a mesh screen.

6. A hook for attaching a surgical rod to a vertebra, comprising:
a rod-receiving portion including a pair of side walls that form a channel for receiving the surgical rod; and
a bone receiving portion extending distally from the rod-receiving portion and forming a hook, the hook including at least one flow path for enhanced flow of bone material through the hook during bone fusion.
